(19) United States
(12) Patent Application Publication
(10) Pub. No.: US 2012/0035684 A1
(43) Pub. Date: Feb. 9, 2012

(54) Multiplexed, Multi-Electrode Neurostimulation Devices with Integrated Circuits Having Integrated Electrodes

(76) Inventors: Todd Thompson, San Jose, CA (US); Mark Zdeblick, Portola Valley, CA (US); Angela Strand, Portland, OR (US); Marc Jensen, Los Gatos, CA (US)

(21) Appl. No.: 13/146,896

(22) PCT Filed: Feb. 9, 2010
(86) PCT No.: PCT/US10/23687

§ 371 Date: Oct. 17, 2011

Related U.S. Application Data
(60) Provisional application No. 61/151,171, filed on Feb. 9, 2009.

(51) Int. Cl. A61N 1/372; H01L 21/283

(52) U.S. Cl. 607/59; 607/72; 438/669; 257/E21.159

(57) ABSTRACT

Implantable stimulation devices are provided. Aspects of the devices include a multiplexed multi-electrode component configured for neural stimulation. The multiplexed multi-electrode component includes two or more individually addressable satellite electrode structures electrically coupled to a common conductor. The satellite structures include a hermetically sealed integrated control circuit operatively coupled to one or more electrodes. Also provided are methods of manufacturing wherein the application of laser welding is avoided in forming the satellite electrode structures and an integrated control circuit thereof is thereby shielded from mechanical stress during satellite manufacture. Additionally provided are systems that include the devices of the invention, as well as methods of using the systems and devices in a variety of different applications.

### RELATED APPLICATION AND CROSS REFERENCE

This application claims the benefit of U.S. Provisional Patent Application No. 61/151,171, filed on Feb. 9, 2009, titled “Multiplexed, Multi-Electrode Neurostimulation Devices with Integrated Circuits Having Integrated Electrodes”, which application is incorporated by reference in its entirety for all purposes in the Present Application.

### FIELD OF THE INVENTION

The present invention is related to electrical devices and systems for electrical stimulation of living mammalian tissue and, more specifically, to implantable electrical leads that include satellite structures, wherein each satellite structure controllably delivers the electrical stimulation to tissue and each satellite structure includes a controller device coupled with one or more electrodes.

### BACKGROUND

Implantable neurostimulators are used to deliver neurostimulation therapy to patients to treat a variety of symptoms or conditions such as chronic pain, tremor, Parkinson's disease, epilepsy, incontinence, or gastroparesis. Implantable neurostimulators may deliver neurostimulation therapy in the form of electrical pulses via implantable leads that include electrodes. To treat the above-identified symptoms or conditions, implantable leads may be implanted along nerves, within the epidural or intrathecal space of the spinal column, and around the brain, or other organs or tissue of a patient, depending on the particular condition that is sought to be treated with the device.

The length of the effective lifespan of implanted leads affects the benefit derived by a host patient. Replacing leads and leads components after implantation is generally to be avoided as increased costs and undesirable complications may arise when removing implanted leads or elements thereof is required. Implantable leads that demonstrate longer effective lifespan offer fewer health risks and are thereof of more benefit to the patient.

Various implantable lead designs may have different shapes, to include those leads that are commonly known as paddle leads and percutaneous leads. Paddle leads, which are typically larger than percutaneous leads, are directional and often utilized due to desired stimulus effect on the tissues or areas. Leads include several elements such as conductors, electrodes and insulators may be combined to produce a lead body. A lead may include one or more conductors extending the length of the lead body from a distal end to a proximal end of the lead. The conductors electrically connect one or more electrodes at the distal end to one or more connectors at the proximal end of the lead. The electrodes are designed to form an electrical connection or stimulus point with tissue or organs. Lead connectors (sometimes referred to as terminals, contacts, or contact electrodes) are adapted to electrically and mechanically connect leads to implantable pulse generators or RF receivers (stimulation sources), or other medical devices. An insulating material may form the lead body and surround the conductors for electrical isolation between the conductors and for protection from the external contact and compatibility with a host body.

Such leads may be implanted into a body at an insertion site and extend from the implant site to the stimulation site (area of placement of the electrodes). The implant site may be a subcutaneous pocket that receives and houses the pulse generator or receiver (providing a stimulation source). The implant site may be positioned a distance away from the stimulation site, such as near the buttocks or other place in the torso area. One common configuration is to have the implant site and insertion site located in the lower back area, with the leads extending through the epidural space in the spine to the stimulation site, such as middle back, upper back, neck or brain areas.

There is a long-felt need to provide improved methods and systems that deliver neuroelectrical stimulation to living tissue and increase the effective lifespan of implanted electrical stimulation leads and elements thereof.

### SUMMARY

Implantable electrical stimulation devices are provided. Aspects of the devices include a multiplexed multi-electrode lead configured for neural stimulation. The multiplexed multi-electrode lead includes two or more individually addressable satellite electrode structures electrically coupled to a common conductor. Each satellite structure includes one or more integrated control circuits operatively to one or more electrodes of the comprising satellite structure. One or more electrodes may be formed via a direct conducting path from the integrated control circuit.

Also provided are systems that include the devices of the invention, as well as methods of using the systems and devices in a variety of different applications. Additional or alternative aspects of the invention include a multiplexed multi-electrode component configured for deep brain stimulation and/or sensing.

Alternate methods of manufacturing are further provided wherein the application of laser welding is avoided in the forming processes of the satellite electrode structures and an integrated control circuit, whereby the electrode structures and the integrated control circuit are shielded from undergoing mechanical stress imposed by laser welding.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

Such incorporations include U.S. Nonprovisional patent application Ser. No. 12/305,894; PCT Patent Application Serial No. PCT/US2007/014509 titled “IMPLANTABLE MEDICAL DEVICES COMPRISING CATHODIC ARC PRODUCED STRUCTURES” and published as WO/2007/149546; U.S. Nonprovisional patent application Ser. No. 12/305,910 titled “Metal Binary and Ternary Compounds Produced by Cathodic Arc Deposition; PCT Patent Application Serial No. PCT/US2003/039524 published as WO 2004/052182; PCT Patent Application Serial No. PCT/US2005/031559 published as WO 2006/029090; PCT Patent Application Serial No. PCT/US2005/046811 published as WO 2006/069322; PCT Patent Application Serial No. PCT/US2005/046815 published as WO 2006/069323; PCT Patent Application Serial No. PCT US2006/048944 published as WO 2007/075974; and PCT Application Serial No. PCT/US2007/009270 published under publication no. WO/2007/120884.

The publications discussed or mentioned herein are provided solely for their disclosure prior to the filing date of the present application. Nothing herein is to be construed as an admission that the present invention is not entitled to antedate such publication by virtue of prior invention. Furthermore, the dates of publication provided herein may differ from the actual publication dates which may need to be independently confirmed.

### BRIEF DESCRIPTION OF THE FIGURES

FIG. 1 provides a view of percutaneous lead according to an aspect of the invention, where the percutaneous lead includes several individually addressable satellite electrode structures.

FIG. 1A provides an exploded view of an electrode structure of the lead of FIG. 1.

FIG. 2 is a schematic of an electrode satellite structure that includes an integrated control circuit and at least one electrode, or “first electrode”.

FIG. 3 is an illustration of the first electrode and a second electrode separately coupled to first surface of the integrated control circuit.

FIG. 4 is an illustration of a third electrode that is coupled to the entire first surface of the integrated control circuit.

FIG. 5 is an illustration of a plurality of electrodes that are each coupled to the first surface of the integrated control circuit.

FIG. 6 is an illustration of a plurality of electrodes that are each coupled to various surfaces of the integrated control circuit.

FIG. 7 is an illustration of the integrated control circuit having an additional protective metal layer.

FIG. 8 is an illustration of the integrated control circuit of FIG. 7 having an additional insulative layer.

FIG. 9 is an illustration of the integrated control circuit of FIG. 7 encompassed by an additional hermetically sealing layer.

FIG. 10 is an illustration of the integrated circuit components of the satellite structures including the integrated control circuit positioned with a semiconductor holder and partially encased in an epoxy and a solid support comprising a lead frame.

FIG. 11 is a schematic diagram of an alternate integrated control circuit, or “module”, of FIG. 2.

FIG. 12 is a schematic diagram of a cuff electrode satellite.

FIG. 13A is a perspective view of a band electrode coupled with the integrated control circuit of FIG. 2 or the alternate integrated control module of FIG. 11 and the lead of FIG. 1.

FIG. 13B is a cut-away front view of the band electrode of FIG. 13A coupled with the integrated control circuit of FIG. 2.

FIG. 13C is a schematic diagram of a paddle electrode satellite structure.

FIG. 14 is a schematic diagram of a suite of manufacturing equipment useful for fabricating the lead of FIG. 1, the satellite of FIG. 2, alternate integrated control module of FIG. 11, the cuff electrode satellite of FIG. 12, the band electrode of FIG. 13A and FIG. 13B, and the paddle electrode of FIG. 13C.

FIG. 15 is a process chart of a effective uses of the suite of manufacturing equipment for the fabrication of the lead of FIG. 1, the satellite of FIG. 2, alternate integrated control module of FIG. 11, the cuff electrode satellite of FIG. 12, the band electrode of FIG. 13A and FIG. 13B, and the paddle electrode of FIG. 13C.

### DETAILED DESCRIPTION

Implantable neural stimulation devices are provided. Aspects of the devices include a multiplexed multi-electrode component configured for neural stimulation. The multiplexed multi-electrode component includes two or more individually addressable satellite electrode structures electrically coupled to a common conductor. The individually addressable satellite electrode structures include a hermetically sealed integrated control circuit operatively coupled to one or more electrodes. Also provided are systems that include the devices of the invention, as well as methods of using the systems and devices in a variety of different applications.

In further describing various aspects of the invention, devices of the invention are reviewed first in greater detail, followed by a description of systems and methods of using the same in various applications, including neural stimulation applications.

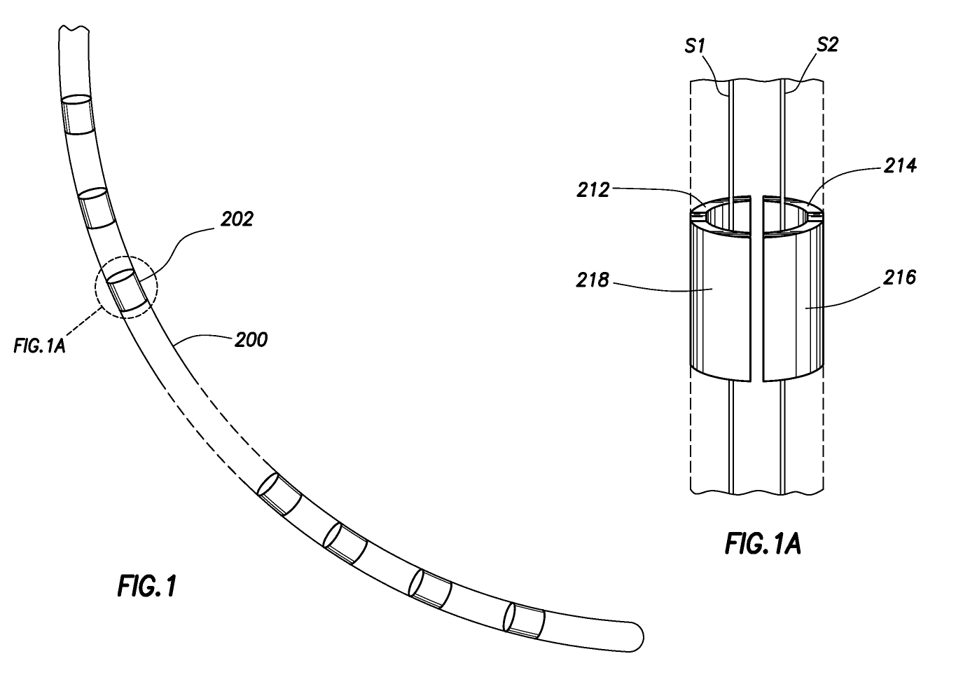

FIG. 1 shows a lead 200 including multiplexed multi-electrode components that are individually addressable satellite structures 202 positioned longitudinally on the lead 200. The lead 200 includes two bus wires S1 and S2, which are coupled to individually addressable electrode satellite structures, such as individually addressable satellite electrode structure 202. FIG. 1A also shows individually addressable satellite electrode structure 202 with an enlarged view. Individually addressable satellite electrode structure 202 includes electrodes 212, 214, 216, and 218, located in the four quadrants of the cylindrical outer walls of satellite 202. FIG. 1B provides a depiction of the arrangement of four electrodes. As indicated above, a given individually addressable satellite electrode structure may include more or less than four electrode elements.

For example, six electrode elements may be present, as shown in FIG. 1C. Each individually addressable satellite electrode structure also contains integrated circuit component inside the structure which communicates with other satellite structures and/or distinct control units, e.g., to receive neurostimulation signals and/or configuration signals that determine which of the different electrodes are to be coupled to bus wires S1 or S2 of FIG. 1A.

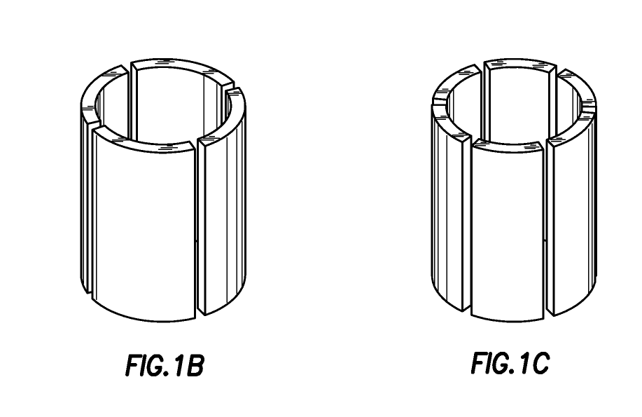

FIG. 2 is a schematic of an electrode satellite structure 202 that includes an integrated control circuit 300 and a first electrode 302. The integrated control circuit 300 includes a control circuitry 304, a selectable current pathway 306, and a first surface 308. The integrated control circuit 300 includes a device communications bus 310 that is coupled to a lead communications bus 312 of the lead 200. The integrated control circuit 300 is operatively coupled with a current switch 314 of the selectable current pathway 306 (hereinafter, “selectable pathway” 306). The integrated control circuit 300 open and closes the current switch 314 in accordance with commands addressed to the comprising electrode satellite structure 202, wherein the commands are received by the control circuit 300 via the device communications bus 310. The integrated control circuit 300 preferably presents a thickness along the Y-axis within the range of ten microns and two hundred fifty microns, and more preferably presents a thickness along the Y-axis within the range of fifty microns and one hundred fifty microns.

The selectable current pathway 306 includes a power bus 316 that is coupled with a common conductor 318 of the lead 200. The common conductor 316, (hereinafter, “power bus” 316) is coupled to a plurality of satellite structures 202 and provides electrical power to each coupled satellite structure 202. The device communications bus 310 is similarly separately coupled to each satellite structure 202 of the plurality of satellite structures 202, wherein commands addressed to individual satellite structures 202 are provided to the plurality of satellite structures 202 via the device communications bus 310.

The substantively hemispherical electrode 302 is coupled to the first surface 308 has a convex shape that extends away from the first surface 308. The substantively hemispherical electrode 302 may preferably have an external diameter of between 0.5 millimeters to 2.0 millimeters, or more preferably an external diameter of between 1.0 millimeters to 1.5 millimeters

The first surface 308 preferably presents a thickness in a Y dimension of from 20 microns to 250 microns. The electrode 302 receives electrical power from the selectable pathway 306 when the current switch 314 is closed as controlled by the control circuit 300. The electrode 302 transfers the electrical power received from the selectable pathway to a target site 320 of an enclosing mammalian tissue environment 322. The roughness of each electrode 302 can range from smooth to a high degree of roughness. The advantage of affecting the performance of the electrode 302 by manufacturing techniques is thereby enabled. One or more electrodes 302 are coated with one or more of various films including, but not limited to, titanium nitride, iridium oxide, and platinum oxide. The film coating of one or more electrodes 302 is preferably within the range of ten angstroms and thirty thousand angstroms. The film coating of one or more electrodes 302 is more preferably within the range of one thousand angstroms and twenty thousand angstroms.

Alternatively, the first electrode 302 may be shaped as a substantively planar sheet having a uniform thickness of a top electrode surface as extending from the first surface 308 measured along the Y-axis. The thickness of the first electrode 302 as measured along the Y-axis and extending from the first surface 308 is preferably within one micron to 250 microns and more preferably from 50 microns to 150 microns. The thickness of the first electrode 302 along the top electrode surface as measured along the Y-axis and from the first surface 308 preferably varies less than 20% and more preferably varies less than 1%.

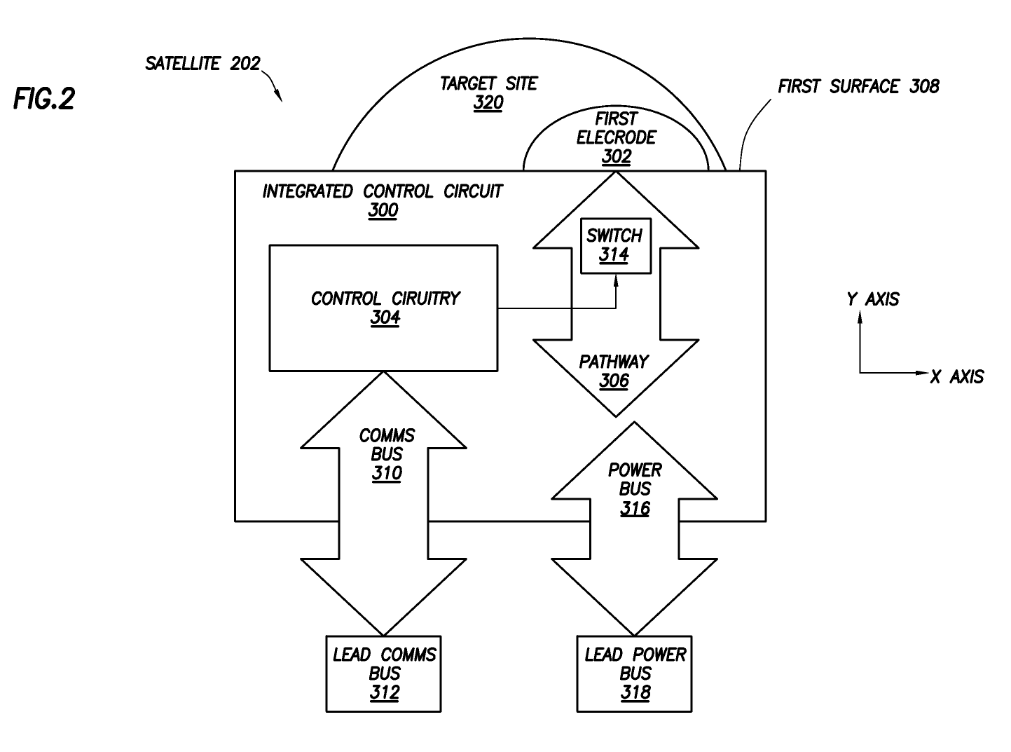

FIG. 3 is an illustration of the first electrode 302 coupled to a first area 324 and a second electrode 326 that is coupled to a second area 328 of the first surface 308 of the integrated control circuit 300. Both the first electrode 302 and the second electrode 326 are coupled to the selectable pathway 306 receive electrical power from the selectable pathway 306 when the current switch 314 is closed as controlled by the control circuit 300. Both the first electrode 302 and the second electrode 326 transfer the electrical power received from the selectable pathway to the target site 320 of the enclosing mammalian tissue environment 322.

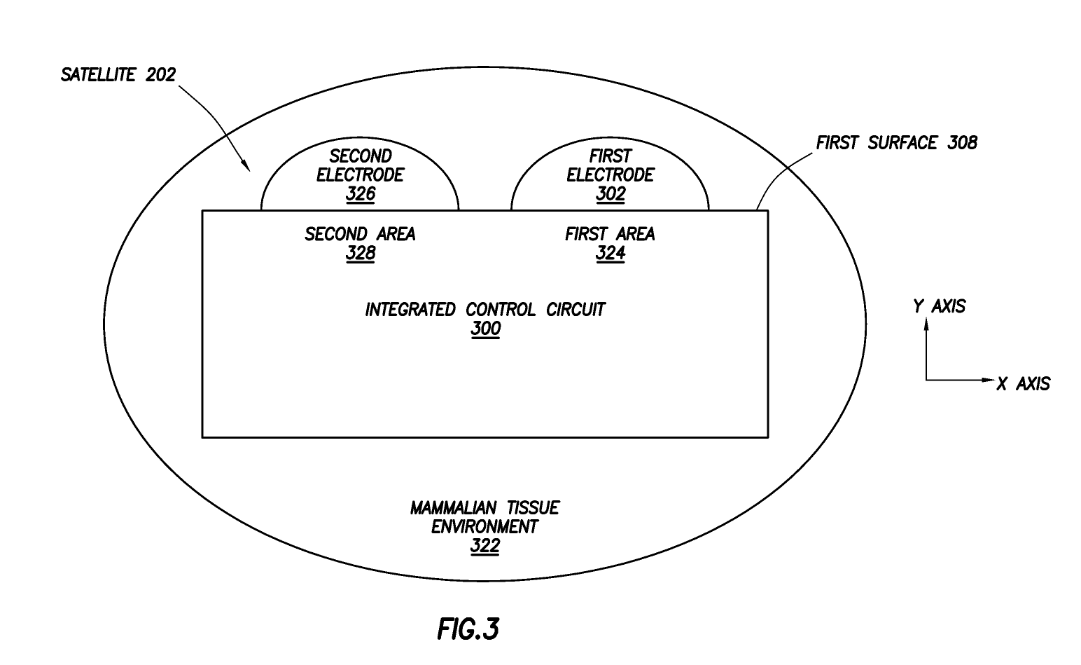

FIG. 4 is an illustration of a third electrode 330 that is coupled to the entire first surface 308 of the integrated control circuit 300. The third electrode 330 may optionally or alternatively extend beyond the first surface 308 and preferably has a thickness in the Y-axis and extending away from the integrated controller 300 for from 20 microns to 300 microns.

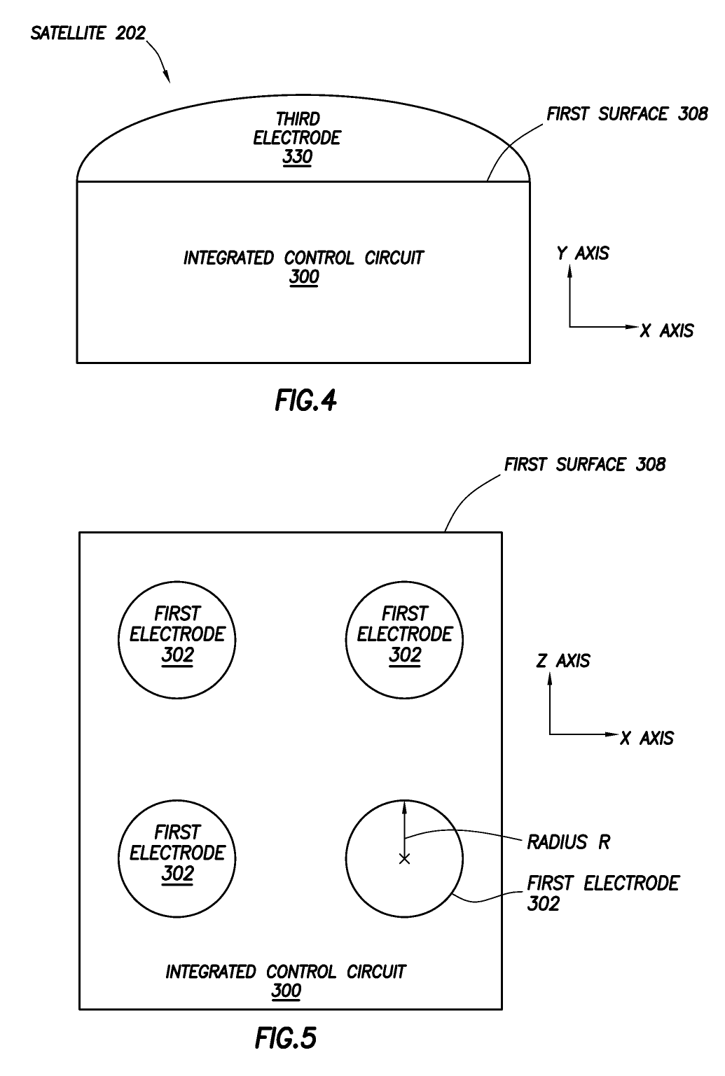

FIG. 5 is a top view illustration of a plurality of first electrodes 302 that are each coupled to the first surface 308 of the integrated control circuit 300. The first electrodes 302 preferably demonstrate a radius R that is within the range of from 5% to 25% of a width along an X-axis or a length along a Z-axis of the first surface 308. Alternatively or additionally, the first electrodes 302 preferably demonstrate a radius R that is within the range of from 5% to 25% of a width along an X-axis or a length along a Z-axis the integrated control circuit 300.

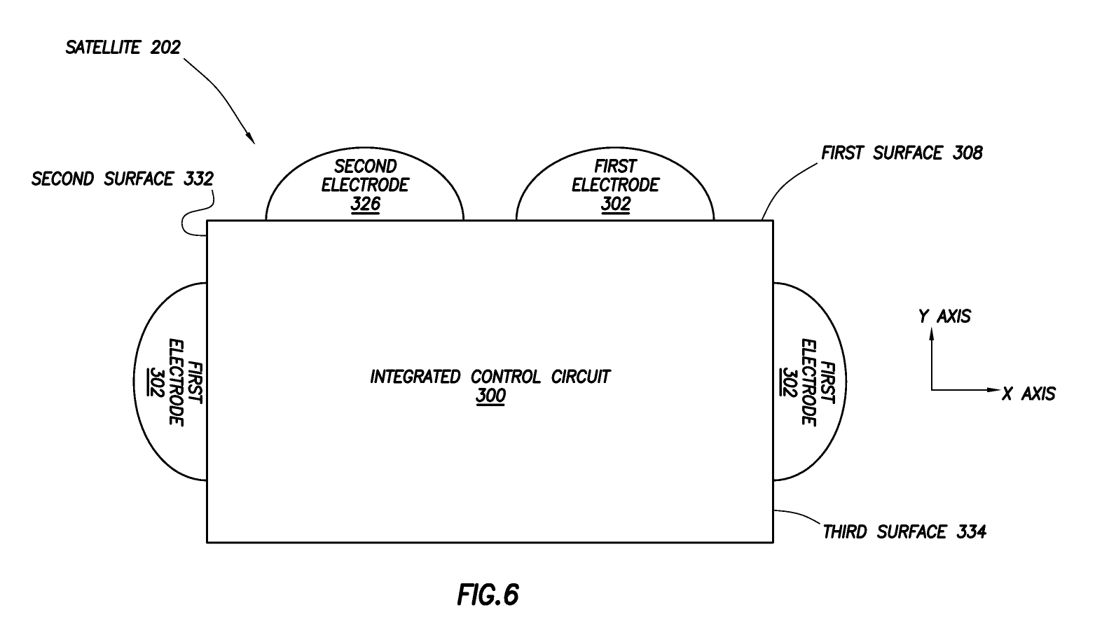

FIG. 6 is an illustration of a plurality of first electrodes 302 that are each coupled to various surfaces 308, 332, and 334 of the integrated control circuit 300. The first surface 308 is substantively orthogonal preferably with less

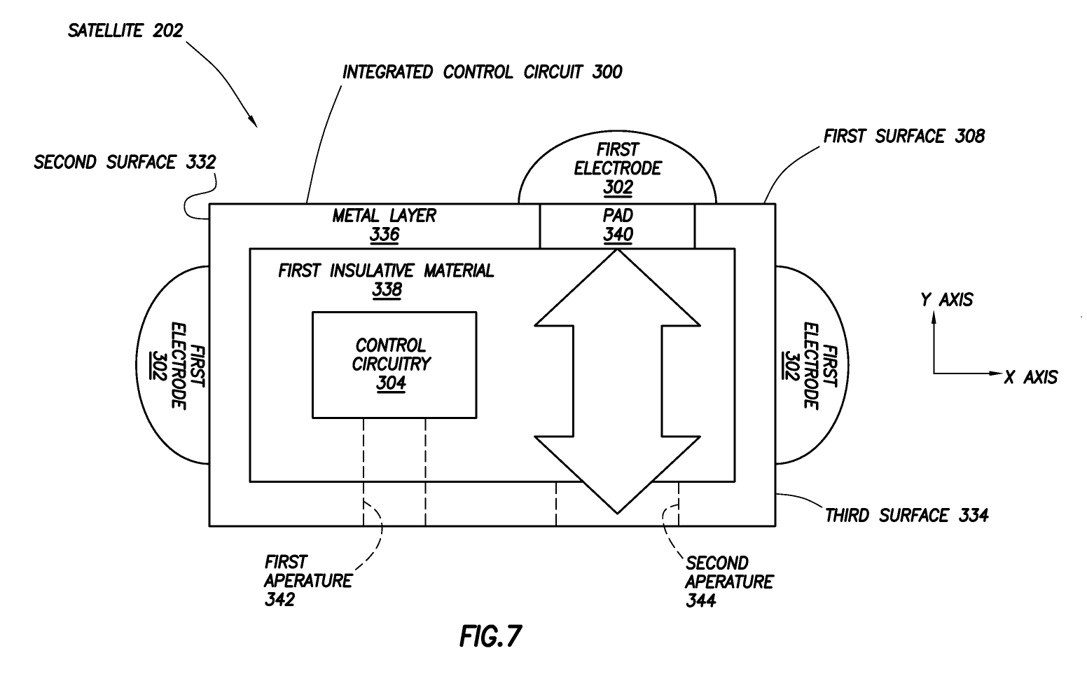

FIG. 7 is an illustration of the integrated control circuit 300 having an additional protective metal layer 336. The metal layer 336 may alternatively, in various alternate configurations of the integrated control circuit 300, extend to (a.) only partially cover the first surface 308, the second surface 332 and/or the third surface 334; (b.) completely cover the first surface 308, the second surface 332 and/or the third surface 334; and (c.) partially or completely encompass the integrated control circuit 300. A first insulative material 338 insulates the control circuit 306 from the metal layer 336, and an electrically conductive electrode pad 340 of the selectable pathway 306 extends through the first insulative material 338 to provide electrical power to one or more electrodes 302, 326 and 330. A first aperture 342 permits the communications bus 310 to operatively couple with the lead communications bus 312 and a second aperture 344 enables the power bus 316 to operatively couple with the common conductor 318.

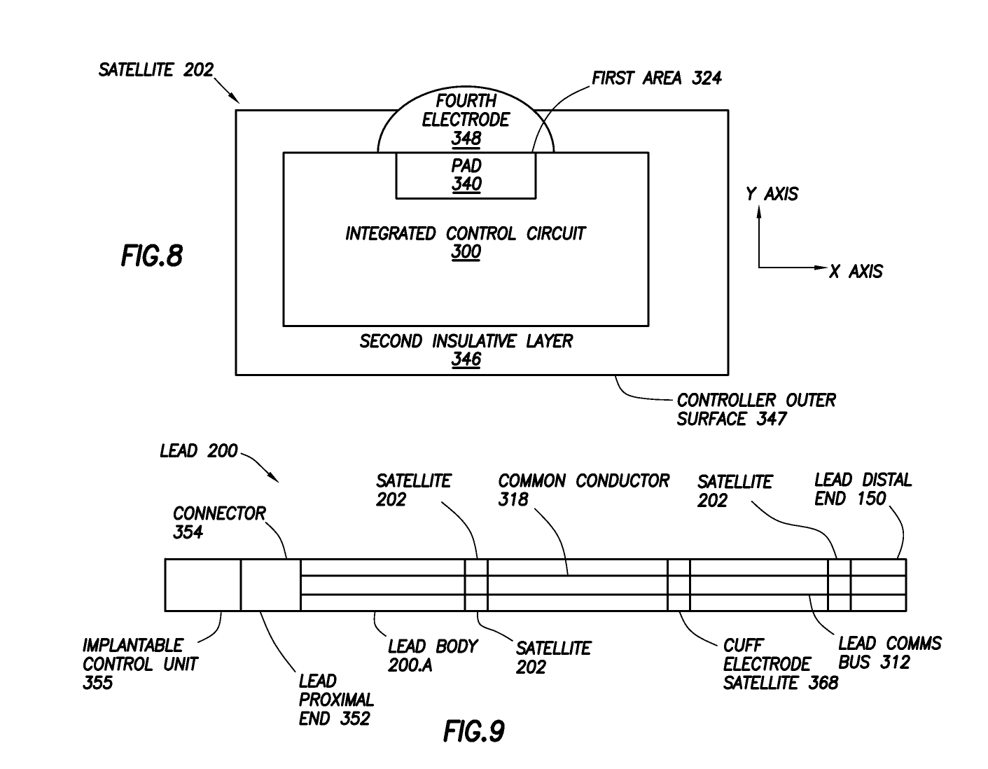

FIG. 8 is an illustration of the integrated control circuit of FIG. 7 having an additional second insulative material 346. A fourth electrode 348 extends through the second insulative layer 346 and makes an operative electrical connection with the electrode pad 340 of the selectable pathway 306. This fourth electrode may be formed by the steps of: (1.) applying a photo resist material layer by photolithography wherein the photo resist material is deposited on the first area 324 of the first face 308; (2.) flowing the material of the second insulative layer 346 in a liquid state over the first face 308 and allowing the liquid material to return to a solid state and thereby form the second insulative layer 346; (3.) removal of the photo resist by wet or dry etch, or other suitable material removal process known in the art; and (4.) depositing a conductive material onto the first area to form the fourth electrode 348. It is understood that other suitable methods known in the art may be used to form and operatively couple the fourth electrode and to the electrode pad 340 and/or the first area.

Individually addressable satellite electrode structures 202 of the leads 200 have hermetically sealed integrated circuit components, such that they include the sealing layer 346 which seals the integrated control circuit 300 from the implanted environment 322 so that the satellite structure 202 maintains functionality, at least for the intended lifespan of the lead 200.

The nature of the sealing layer 346 may vary, so long as it maintains the functionality of the satellite structure 202 in the implanted environment for the desired period of time, such as one week or longer, one month or longer, one year or longer, five years or longer, ten years or longer, twenty-five years or longer, forty years or longer.

In some instances, the sealing layer 346 is a conformal, void-free sealing layer 346, where the sealing layer 346 is present on at least a portion of the outer surface 347 of the integrated control circuit 300 (described above). In some instances, this conformal, void-free sealing layer 346 may be present on substantially all of the outer surfaces of the integrated circuit component. Alternatively, this conformal, void-free sealing layer 346 may be present on only some of the surfaces of the integrated circuit, such as on only one surface or even just a portion of one surface of the integrated circuit component. As such, some sensors have an integrated circuit component completely encased in a conformal, void free sealing layer. Other sensors are configured such that only the top surface of an integrated circuit component is covered with the conformal, void-free sealing layer 346 may be a “thin-film” coating, in that its thickness of the sealing layer along the three orthogonal axes of the Y-axis, X-axis and Z-axis is such that it does not substantially increase the total volume of the integrated circuit structure with which it is associated, where any increase in volume of the structure that can be attributed to the layer may be 10% or less, such as 5% or less, including 1% or less by volume. In some instances, the seal layer 346 has a thickness in a range from 0.1 micron to 10.0 micron, such as in a range from 0.3 micron to 3.0 micron thick, and including in a range 1.0 um thick.

The seal layer 346 may be produced on the integrated circuit component using any of a number of different protocols, including but not limited to planar processing protocols, such as plasma-enhanced-chemical-vapor deposition, physical-vapor deposition, sputtering, evaporation, cathodic-arc deposition, low pressure chemical-vapor deposition.

Additional description of conformal, void-free sealing layers that may be employed for sensors of the invention is provided in PCT application serial no. PCT/US2007/009270 published under publication no. WO/2007/120884, the disclosure of which is herein incorporated by reference.

Also of interest as sealing elements are corrosion-resistant holders 349 having at least one conductive feed-through and a sealing layer; where the sealing layer 346 and the corrosion-resistant holder 349 are configured to define a hermetically sealed container that encloses the integrated control circuit 300. The conductive feed-through may be a metal, such as platinum, iridium, niobium, titanium etc., an alloy of metal and a semiconductor, a nitride, a semiconductor or some other convenient material. In some instances, the corrosion-resistant holder comprises silicon or a ceramic. While dimensions may vary, the corrosion-resistant holder may have walls that are at least one micron thick, such as at least fifty microns thick, where the walls may range from one micron to one hundred twenty-five microns, including from twenty five microns to one hundred microns. Alternatively, the sealing layer 346 may be metallic, where metals of interest include noble metals and alloys thereof, such as niobium, titanium, platinum and platinum alloys. Dimensions of the sealing layer may also vary, ranging in some instances from 0.5 um thick or thicker, such as 2.0 um thick or thicker, and including 20 um thick or thickness, where sealing layer thicknesses may range from 0.5 to 100 um, such as from 1 to 50 um.

In certain configurations, the structure 202 further includes the seal layer 346 present in the hermetically sealed volume. In some cases, the hermetically sealed volume ranges from 1 pl. to 1 milliliter.

In some instances, an in-vivo corrosion-resistant holder 349 is a structure configured to hold the integrated control circuit 300 such that the integrated control circuit 300 is bounded on all but one side by the walls of the corrosion-resistant holder 349. For example, the corrosion-resistant holder 349 may include sidewalls and a bottom, where the holder may have a variety of different configurations as long as it contains the integrated circuit component in a manner such that the component is held in a volume bounded on all but one side.

Accordingly, the shape 349 of the holder may be square, circular, ovoid, rectangular, or some other shape as desired. Additional description of corrosion resistant holders that may be employed for sensors 300.C of the invention is provided in PCT application serial no.

PCT/US2005/046815 published under publication no. WO/2006/069323, the disclosure of which is herein incorporated by reference.

Of particular interest are aspects in which at least one electrode 302 is formed as via a direct conducting path from the integrated control circuit 300. As such, the material(s) forming the electrode 302 may be recessed, convex, or flush with respect to an outer surface of the lead 200. In this manner, economical use of manufacturing materials and processes may be achieved. Further, in various aspects, the overall diameter of the lead 200 may be relatively small, e.g., approximately 0.5 mm to 3.0 mm. In some aspects, the lead diameter may be approximately 1.0 mm to 1.5 mm, or approximately 1.25 mm.

Various aspects may permit use of a guidewire lumen (not shown) of a relatively small dimension. In various aspects, a material may be deposited or otherwise associated with the integrated control circuit 300 to strengthen or otherwise support the integrated control circuit 300 and associated components. The preferable materials to form the support structure 358 include, for example, platinum, platinum iridium, niobium, and titanium. A skilled artisan will appreciate that various other materials and combinations of materials may be employed.

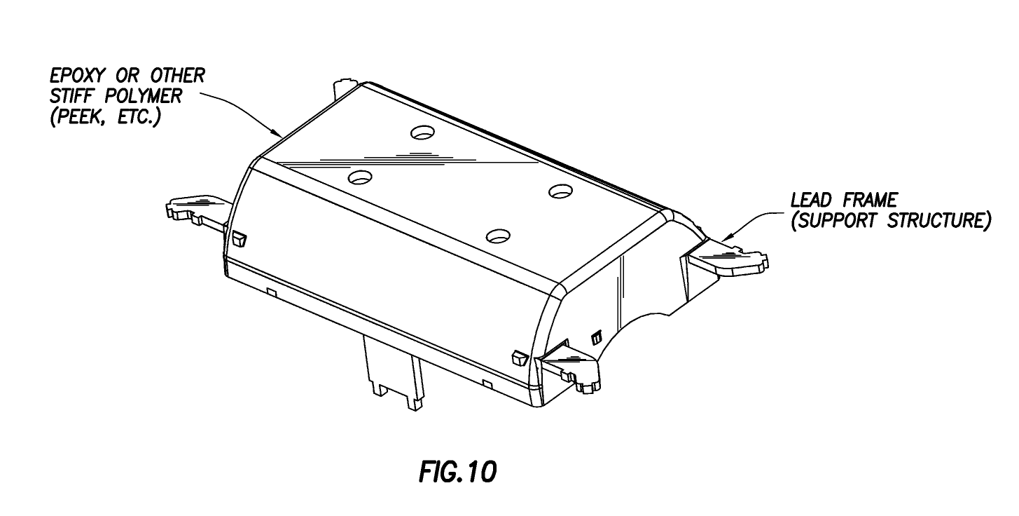

Referring now to FIG. 9, the lead 200 may include one or more lead components, to include a plurality of satellite structures 202. Lead components are elongated structures having lengths that are 2 times or longer than their widths, such as 5 times or longer than their widths, including 10, 15, 20, 25, 50, 100 times or longer than their widths. In certain instances, the leads have lengths of 10 mm or longer, such as 25 mm or longer, including 50 mm or longer, such as 100 mm or longer. A variety of different lead configurations may be employed, where the lead in various aspects is an elongated cylindrical structure having a proximal end 350 and a distal end 352. The proximal end 350 may include a connector element 354, e.g., an IS-1 connector, for connecting to an implantable lead control unit 355, e.g., present in a “can” or analogous device. The lead 200 may include one or more lumens, e.g., for use with a guidewire (not shown), for housing one or more conductive elements, e.g., wires 312 and 318, etc. The distal end of the lead 200 may include a variety of different features as desired, e.g., a securing means. Leads 200 may be fabricated as flexible structures, where the internal common conductor 318 and the lead communications bus 312 may include wires, coils or cables made of a suitable material, such as alloy MP35N (a nickel-cobalt-chromium-molybdenum alloy), platinum, platinum-10 iridium, etc. A lead body 200.A may be any suitable material, such as a polymeric material, including polyurethane or silicone.

Lead components of the invention may have a variety of shapes, as desired. In some instances, the leads 200 have a standard percutaneous shape, as found in conventional percutaneous neural stimulation leads. In some instances, the leads have a standard paddle shape, as found in conventional paddle neural stimulation leads.

Devices of invention include a multiplexed multi-electrode component. Multiplexed multi-electrode components include two or more electrodes 302 which are electrically coupled, either directly or through the selectable pathway 306, to the common conductor 318 or set of common conductors 318, such that the two or more electrodes 302 share one or more conductors 318. The term “conductor” refers to a variety of configurations of electrically conductive elements, including wires, cables, etc. A variety of different structures may be implemented to provide the multiplex configuration. Multiplex configurations of interest include, but are not limited to, those described in: PCT application no. PCT/US2003/039524 published as WO 10 2004/052182; PCT application no. PCTI US2005/031559 published as WO 2006/029090; PCT application no. PCTI US2005/046811 published as WO 2006/069322; PCT application no. PCTI US2005/046815 published as WO 2006/069323; and PCT application no. PCT US2006/048944 published as WO 2007/075974; the disclosures of which are herein incorporated by reference. The multiplexed multi-electrode components include two or more individually addressable satellite electrode structures 202. In some instances, more than two individually addressable satellite structures 202 are present in the device, such as three or more, four or more, five or more, six or more, ten or more, twenty or more (including twenty-four), thirty or more, fifty or more, etc. Individually addressable satellite electrode structures 202 are those that can be individually controlled from a site remote from the satellite electrode structure 202, such as a separate implanted control unit to which the device is operatively coupled or to an extracorporeal control unit. Satellite electrode structures 202 are structures that include an integrated circuit control device 300 and at least one electrode element 302. The satellite electrode structures 202 of the invention include control circuitry 304 in the form of an integrated circuit that imparts addressability to the satellite electrode structure.

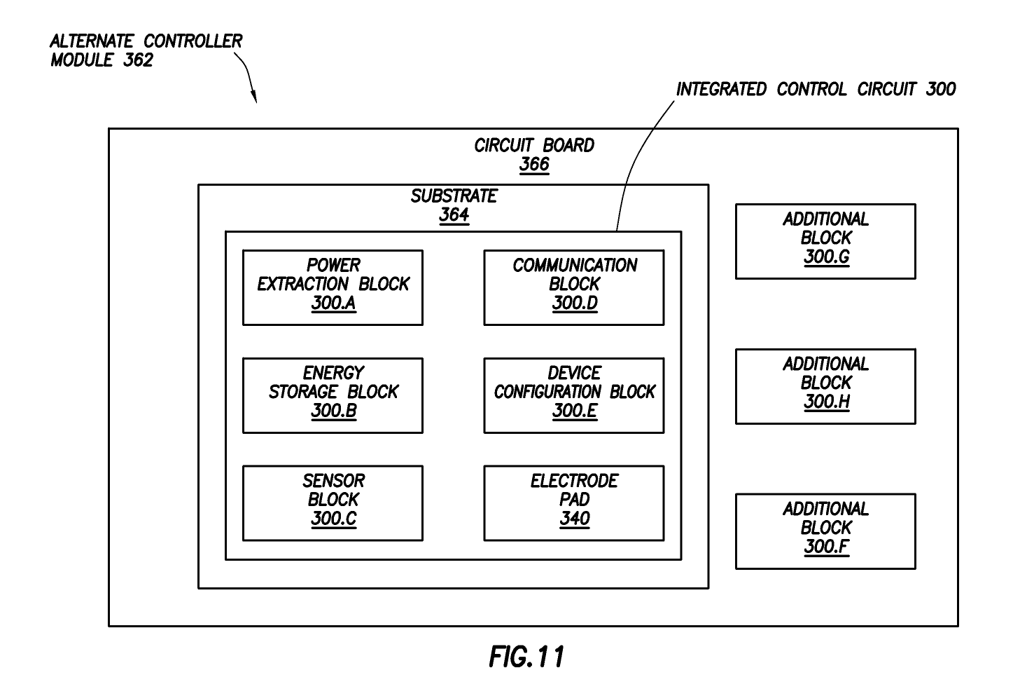

Referring now to FIG. 10, integrated circuit components of the satellite 202 structures are constructs that include the integrated control circuit 300 positioned with the corrosion-resistant holder 349 and partially encased in an epoxy 356 and a solid support 358 comprising a lead frame 360. In variations of the invention, the solid support 358 may be small, for example where it is dimensioned to have a width ranging from 0.01 mm to 100 mm, such as from 0.1 mm to 20 mm, and including from 0.5 mm to 2 mm; a length ranging from 0.01 mm to 100 mm, such as from 0.1 mm to 20 mm, and including from 0.5 mm to' 2 mm, and a height ranging from 0.01 mm to 10 mm, including from 0.05 mm to 2 mm, and including from 0.1 mm to 0.5 mm.

The satellite structure 202 may take a variety of different configurations, such as but not limited to: a chip configuration, a cylinder configuration, a spherical configuration, a disc configuration, or other suitable configuration known in the art. A particular configuration may be selected based on intended application and/or method of manufacture. While the material from which the solid support 358 is fabricated may vary considerably depending on the particular lead 200 for which the satellite structure 202 is configured for use. The preferable materials to form the solid support 358 as an electrically conductive element include, for example, platinum, platinum iridium, niobium, and titanium. In certain instances when it is desirable that the solid support 358 be partially or wholly electrically insulating, the solid support 358 may be made up in whole or in part of an insulative material, such as silicone, polyurethane, urethane co-polymers, or various other materials and combinations of materials.

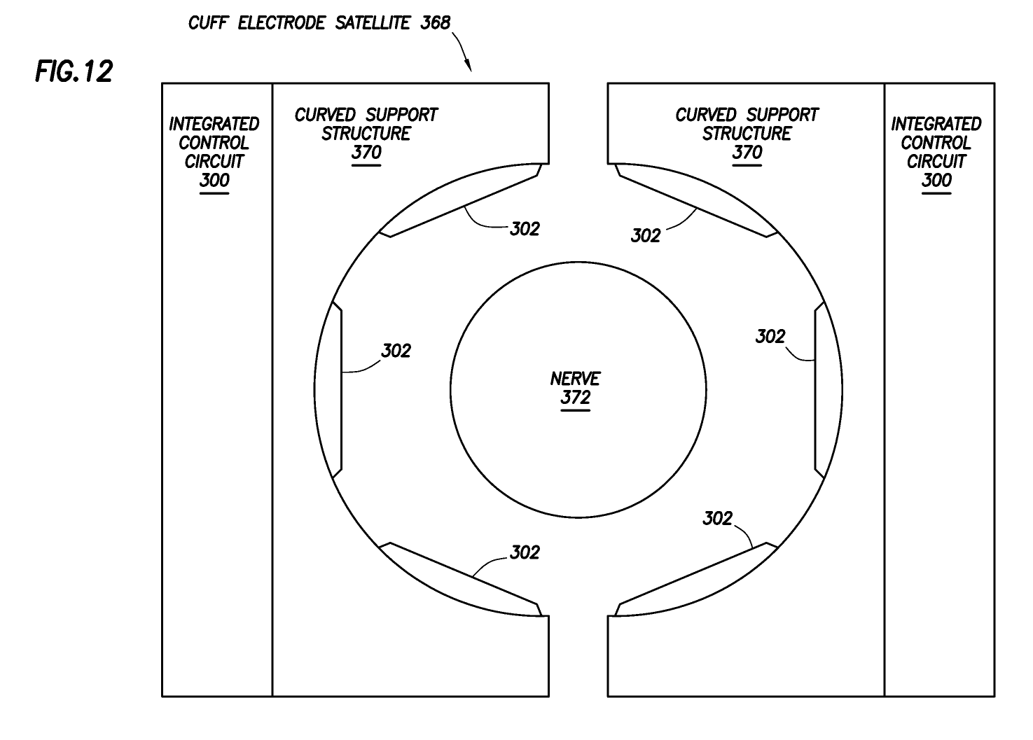

Referring now to FIG. 11, the integrated circuit components of the individually addressable satellite electrode structures 202 may include a number of distinct functional blocks, i.e., modules. An alternate controller module 362 of the satellite structure 202 is provided that includes the integrated control circuit 300. The integrated control circuit 300 is positioned on a substrate 364, and the substrate 364 is coupled with a circuit board 366 of the alternate controller module 362. The substrate 364 may comprise a semiconductor material, such as a silicon wafer. The integrated control circuit 300 may, and/or the alternate controller module 362 alternatively or additionally may, include a number of distinct functional circuitry blocks 300.A-300.H, or “blocks” 300.A-300.H. In some instances, the alternate controller module 362, or alternatively or additionally the integrated control circuit 300, includes at least the following functional blocks: a power extraction functional block 300.A; an energy storage functional block 300.B; a sensor functional block 300.C; a communication functional block 300.D; and a device configuration functional block 300.E. The alternate controller module 362 may further include additional blocks 300.F-300.H.

The power extraction block 300.A is coupled with the common conductor 318 and directs received electrical energy to the electrode 302 via the electrode pad 340 and alternatively for storage in the energy storage block 300.B. It is understood that the alternate controller module 362 and/or the integrated control circuit may comprise a plurality of electrode pads 340. The sensor block 300.C, or “sensor” 300.C, provides a biological parameter detection or measurement capability to the integrated control circuit 300, wherein detections or measurements generated by the sensor block 300.C are transmitted to an implantable control unit and/or the extracorporeal control unit via the communication block 300.D. The communication block 300.D is communicatively coupled with the lead communications bus 312 and is communicatively there through to the implantable control unit and/or the extracorporeal control unit. The communication block 300.D further provides programming instructions and data received via the power and signal bus 109 to the device configuration block 300.E.

Within a given satellite electrode structure 202, at least some of, e.g., two or more, up to and including all of, the functional blocks 300.A-300.H may be present in the single integrated control circuit 300. By single integrated circuit is meant a single circuit structure that includes all of the different desired functional blocks for the invented satellite 202. In these types of structures, the integrated control circuit 300 is a monolithic integrated circuit that is a miniaturized electronic circuit which may be made up of semiconductor and passive components that have been manufactured in the surface of a thin substrate 364 of semiconductor material. Sensor blocks 300.C of the invention may also include integrated circuits that are hybrid integrated circuits, which are miniaturized electronic circuits constructed of individual semiconductor devices, as well as passive components, bonded to the substrate 364 or the circuit board 364.

Within a given satellite electrode structure 202, at least some of, e.g., two or more, up to and including all of, the functional blocks 300.A-300.H may be present in the integrated control circuit 300 as a single integrated circuit. By single integrated circuit is meant a single circuit structure that includes all of the different desired functional blocks for the device. In these types of structures, the integrated control circuit 300 is a monolithic integrated circuit that is a miniaturized electronic circuit which may be made up of semiconductor and passive components that have been manufactured in the surface of the thin substrate 354 of semiconductor material.

Sensors 300.C of the invention may also include integrated circuits that are hybrid integrated circuits, which are miniaturized electronic circuits constructed of individual semiconductor devices, as well as passive components, bonded to the substrate 364 or the circuit board 366.

A given satellite electrode structure 202 may include a single electrode element 302 operatively associated with an integrated control circuit 300, or two or more electrodes 302 operatively associated with the same integrated control circuit 300, such as three or more electrodes 302, four or more electrodes 320, six or more electrodes 302, or a plurality of electrodes 302. In various aspects, the satellite structure 202 includes two or more electrode elements 202, such as three or more electrode elements 202, including four or more electrode elements 302, or a plurality of electrodes 302, wherein the satellite structure 202 is a segmented electrode structure. The various electrode elements 302 may be positioned in three-dimensional space relative to their integrated control circuit 300 to which the electrode elements 302 are electronically associated in a number of different ways. For example, the multiple electrodes 302 may be radially distributed, i.e., axially uniformly positioned, about the integrated control circuit 300. Alternatively, the multiple electrodes 302 may be positioned to a first surface 308 of integrated control circuit 300.

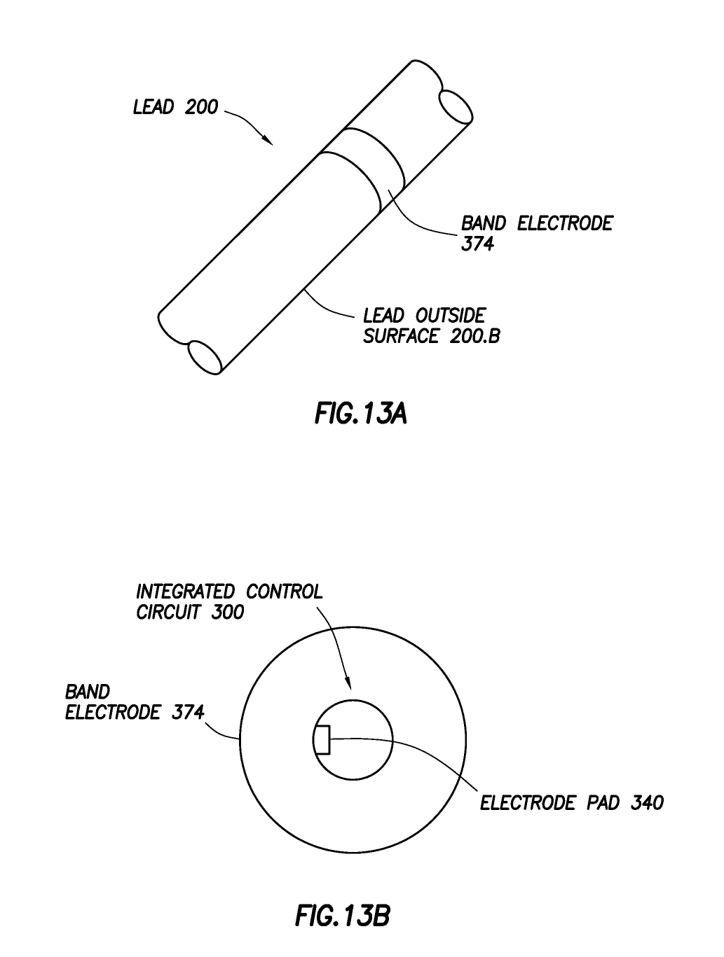

Referring now to FIG. 12, a cuff electrode 368 that may be comprised within the lead 200. The cuff electrode device 368 includes integrated control circuits 300, partially curved support structures 370, and one or more electrodes 302. In various aspects, the at least one curved support structure 370 may be mechanically associated with the integrated control circuits 300, or be independent, integrated, or partially integrated support structures 370. The curved support structure 370 may be formed from various materials, e.g., platinum, platinum-iridium, etc. The one or more electrodes 302 may be disposed on at least one inside surface of the curved support structure 370, such that the electrodes 302 contact targeted tissue, e.g., the vagus nerve 372. The support structure 370 and/or overall construction of the cuff electrode device 368 may facilitate avoidance of stimulation of untargeted tissue, e.g., the voice box. Such form factors include curved support structures 370 forming an aperture therein, e.g., “cuff-shaped”, “clamshell-shaped”, etc. Various components and combinations of components may be similar to above-described multiplexed, multi-electrode device, facilitating stimulation and/or sensing of targeted tissue areas.

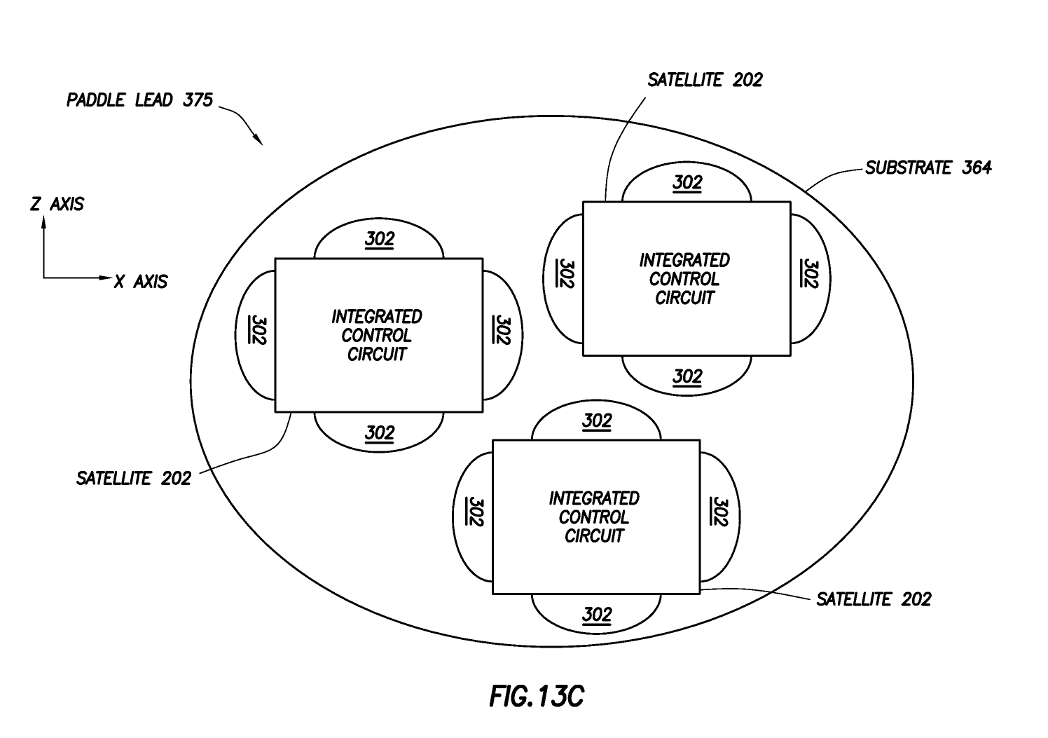

Referring now to FIG. 13A, FIG. 13B and FIG. 13C, in a still additional example, the electrode 302 is formed by physically attaching a predetermined structure with respect to the integrated control circuit 300 and may be attached, for example, to the first surface 302, of the integrated control circuit 300 such that a direct conducting path is formed from the electrode contact pad 340 of the integrated control circuit 300. In this manner, the method of the present invention provides for many stimulation locations with significantly less complexity than hard-wired approaches.

FIG. 13A is an isometric view of the surface band-type electrode 374 attached to the lead 200 and contacting the integrated control circuit 300, and FIG. 13B is a cut-away side view of the surface band-type electrode 374 attached to the lead 200 and contacting the first surface 308 of the integrated control circuit 300, or alternatively the alternate integrated control controller module of FIG. 11. The surface band-type electrode 374 may reside in parallel with a lead outside surface 200.B of the lead body 200.A.

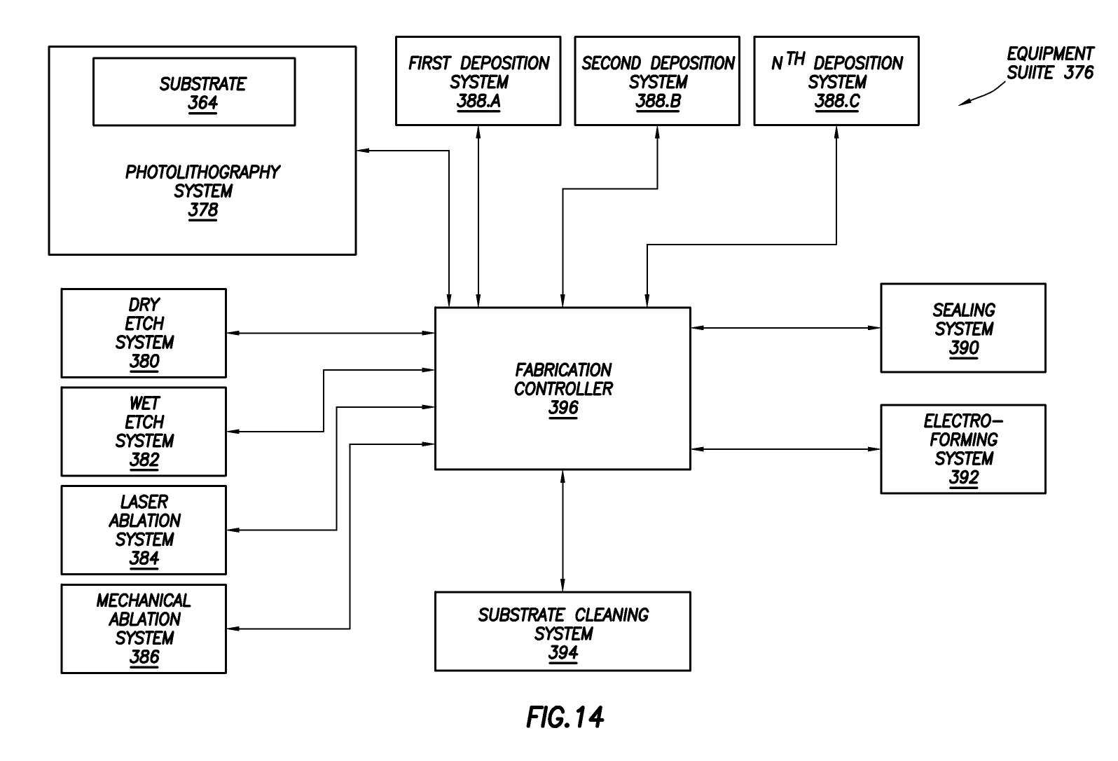

FIG. 13C shows a paddle lead 375 that includes multiple individually addressable satellite electrode structures 202. Underlying the shown electrode structures 202 may be a laser cut pattern of conductive elements as described above. As shown, all of the electrode structures 202 of the paddle 375 are coupled to two wires S1 and S2 such that the paddle lead 375 has a multiplexed configuration.

[Referring now to FIG. 14, FIG. 14 illustrates an equipment suite 376 of electronic device and semiconductor fabrication equipment useful in partially or wholly manufacturing the lead 200, the invented device 202, to include the cuff device 368, as well as components thereof, such as the electrodes 302, the lead communications bus 312, the lead common conductor 318, and the satellites 202. One or more photolithography systems 378 enable the positioning of photoreseist material onto the substrate 364. One or more dry etch systems 380 and wet etch systems 382 are used to remove exposed material from the substrate 364. A laser ablation system 38 and/or a mechanical ablation system 386, e.g., a milling system are used to ablate material from the substrate 364. One or more deposition systems 388.A-388.N, e.g., sputtering systems, liquefied material deposition systems, are used to deposit materials, such as the insulative material 338 and 346, the corrosion-resistant holder 349, the metal protection layer 336, onto the substrate 364, into the satellite 202, or to form the satellite 202. One or more sealing systems 390 applies and secures the sealing layer 346 as part of the satellite 202. An electro-forming system 392 causes the electrode 102 to establish an electrical connection with the electrode contact pad 340. A substrate cleaning system 394 removes debris and excess, unwanted contamination from the satellite 202. A fabrication controller 396 is an information technology system that may be communicatively coupled to one or more equipments 378-394 to manage the whole or partial fabrication of the lead 200, one or more satellites 202, to include the cuff device 176, as well as components thereof, such as the electrodes 1302, the lead communications bus 312, and the lead common conductor 318.

One or more deposition systems 388.A-388.N are used to implement deposition techniques in certain aspects of fabrication of one or more leads 200, satellites 202, and electrodes 302 devices or components thereof include, but are not limited to: electroplating, cathodic arc deposition, plasma spray, sputtering, e-beam evaporation, physical vapor deposition, chemical vapor deposition, and plasma enhanced chemical vapor deposition. Material removal techniques of interest include, but are not limited to: reactive ion etching, anisotropic chemical etching, isotropic chemical etching, planarization, e.g., via chemical mechanical polishing, laser ablation, electronic discharge machining (EDM), etc. Also of interest are lithographic protocols. Of interest in certain aspects is the use of planar processing protocols, in which structures are built up and/or removed from a surface or surfaces of an initially planar substrate using a variety of different material removal and deposition protocols applied to the substrate in a sequential manner.

In particular, The roughness of each electrode 302 can range from smooth to a high degree of roughness by variable applications of the equipment suite 376. The advantage of affecting and improving the performance of the electrode 302 by manufacturing techniques is thereby enabled.

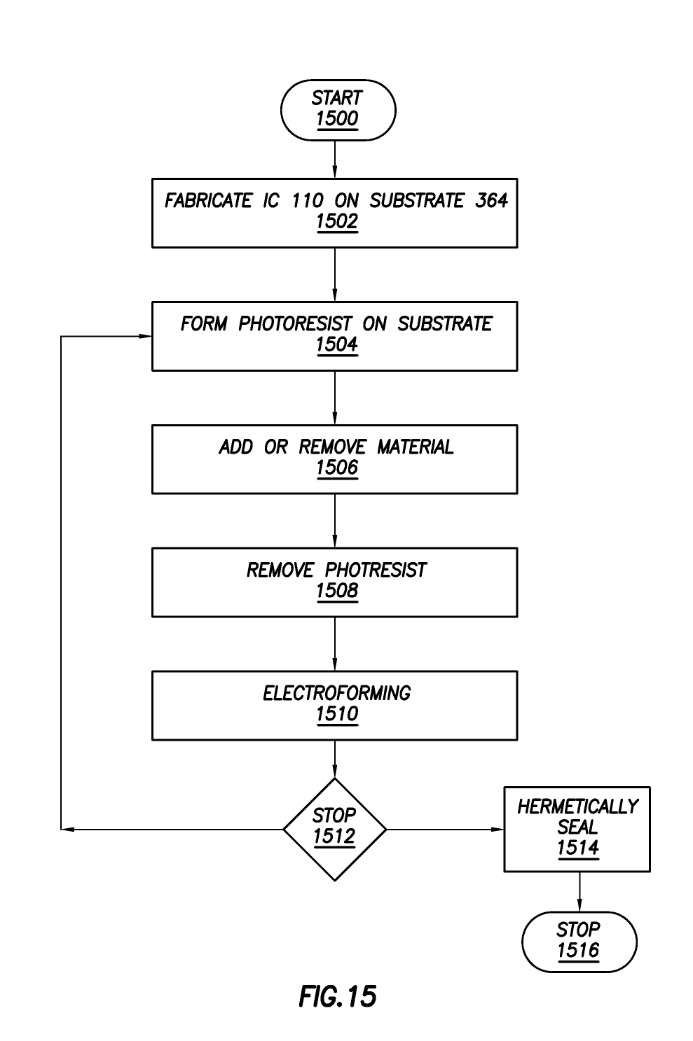

Referring now to FIG. 15, FIG. 15 is a process chart in accordance with the method of the present invention for to apply the equipment suite 376 to wholly or partially manufacture the lead 200, one or more satellites 202, to include the cuff device 368 and the paddle lead 375, as well as components thereof, such as the electrodes 302, the lead communications bus 312, and the lead common conductor 318. In optional step 1502 the integrated control circuit 300 is fabricated by standard electronic and semiconductor device manufacturing methods known in the art, wherein the integrated control circuit 300 may optionally be attached to the substrate 364. In step 1504 photoresist is added to the substrate 364 and/or the circuit board 366. In step 1506 material is removed or alternatively added to the integrated control circuit 300 and/or the circuit board 366, wherein the material may be or at least partially form, for example, the support structure 370, one or more functional blocks 300.A-1300.F, the satellite 202, the electrode 302, the lead 200, the electrode contact pad 340, the sealing layer 346, the first insulative material 338, the integrated circuit holder 349, and the metal protection layer 336. In optional step 1508 the photoresist is removed. In optional step 1510 the electro-forming system 392 is applied to effectuate electro-forming within the lead 200 and/or one or more satellites 202. In step 1512 a human operator or the fabrication controller 396 determines whether to (a.) continue the manufacturing process by repeating a variation of the cycle of steps 1504 through 1512, or to (b.) proceed on to step 1514 and to apply the sealing system 1390 to hermetically seal part or all of the lead 200, the cuff electrode 368, the paddle lead 375 and/or one or more satellites 202, well as components thereof, such as the lead common conductor 318 and the lead communications bus 312.

Methods of manufacturing the lead 200, the satellites 200, 368 and the electrodes 302 and 374 are further provided wherein the application of laser welding is avoided in forming and assembling the lead 200, the satellite electrode structures 202 and electrodes 302 and 374. The lead 200, the satellite electrode structures 202 and electrodes 302 and 374 are thereby shielded from undergoing mechanical stress imposed by a laser welding process.

In a first example, the electrode 302 is formed by exposure of the thick metal protection layer 336 of the integrated control circuit 300 such that a direct conducting path is formed from the electrode contact of the integrated control circuit 300. In this manner, the electrode 302 provides for many stimulation locations with significantly less complexity than hardwired approaches.

In a second example, the fourth electrode 348 is formed by blocking the flow of the second insulative material 346 to the conductive material of the electrode connection of the integrated control circuit 300. In this manner, the fourth electrode 348 provides for many stimulation locations with significantly less complexity than hard-wired approaches.

In a third example, the electrode is formed by creating electrodes, e.g., “posts”, at various predetermined positions with respect to the integrated control circuit 300.

Such positions include over the metal layer 336 lay of the integrated circuit such that a direct conducting path is formed from the electrode contact of the integrated control circuit 300. In this manner, the first electrode 302 provides for many stimulation locations with significantly less complexity than hard-wired approaches.

In a fourth example, the first electrode 302 is formed by physically attaching a predetermined structure with respect to the integrated control circuit 300. Such structures include a surface band-type electrode and may be attached, for example, to the first surface 308 of the integrated control circuit 300 such that a direct conducting path is formed from the electrode contact pad 340 of the integrated control circuit 300. In this manner, the first electrode 302 provides for many stimulation locations with significantly less complexity than hardwired approaches.

In a fifth example, the first electrode 302 is formed via a milling technique such as mechanical or laser ablation, that a direct conducting path is formed from removing the second insulative material 346 above the first are 324 of the first surface 308 on the integrated circuit. In this manner, the first electrode 302 provides for many stimulation locations with significantly less complexity than hard-wired approaches.

In a sixth example, the electrode 302 is formed via electroforming or similar suitable techniques known in the art such that a direct conducting path is formed from the electrode contact pad 340 of the integrated control circuit 300. In this manner, the first electrode 302 provides for many stimulation locations with significantly less complexity than hard-wired approaches.

In a seventh example, the electrode 302 is formed by suitable deposition techniques and systems including, but not limited to cathodic arc deposition, electroplating, plasma spray, sputtering, e-beam evaporation, physical vapor deposition, chemical vapor deposition, and plasma enhanced chemical vapor deposition.

Any of a variety of different protocols may be employed in manufacturing the elements and devices of the invention. For example, molding, deposition and material removal, planar processing techniques, such as Micro-Electro-Mechanical Systems (MEMS) fabrication, may be employed. Deposition techniques that may be employed in certain aspects of fabrication of the devices or components thereof include, but are not limited to: electroplating, cathodic arc deposition, plasma spray, sputtering, e-beam evaporation, physical vapor deposition, chemical vapor deposition, plasma enhanced chemical vapor deposition, etc. Material removal techniques of interest include, but are not limited to: reactive ion etching, anisotropic chemical etching, isotropic chemical etching, planarization, e.g., via chemical mechanical polishing, laser ablation, electronic discharge machining (EDM), etc. Also of interest are lithographic protocols. Of interest in certain aspects is the use of planar processing protocols, in which structures are built up and/or removed from a surface or surfaces of an initially planar substrate using a variety of different material removal and deposition protocols applied to the substrate in a sequential manner.

In some instances, laser cut wires are employed as conductive elements for devices of the invention, such as for conductive elements of lead elements of devices of the invention. For example, conductive elements may be laser cut from a single sheet of metal. The pattern of the laser cut conductive elements may be chosen to match the positioning of the individually addressable satellite electrode structures of the lead. In this manner, the conductors and electrode structures may be aligned and then overlaid with the appropriate polymeric material to produce the desired lead structure. The laser cut conductive elements may have a variety of configurations from linear to curvilinear, sinusoidal or other fatigue resistance configuration. Instead of laser cutting, the conductor could also be fabricated using a deposition protocol, such as described above.

Devices of the invention may be implanted using any convenient protocol. Standard implantation procedures for percutaneous and paddle leads may be adapted for implantation of devices of the invention. The devices may be configured for ease of implantation. For example, devices may include a deployable element, such as lead components that inflate, e.g., with a gas or suitable liquid medium, to assume a desired configuration.

Also provided are systems that include one more neural stimulation devices as described above operatively coupled to an implantable controller, which may be an implantable pulse generator. The implantable controller may be any suitable controller, including any of a number of implantable pulse generators currently employed for neurostimulation procedures, where the devices may be modified as desired to work with multiplexed multi-electrode neurostimulation devices of the invention. Also part of the systems may be any number of additional components, as desired, including extra-corporeal control units configured to transmit data and/or power to and/or receive data from the implantable components.

Also provided are methods of using the systems of the invention. The methods of the invention generally include: providing a system of the invention, e.g., as described above, that includes an implantable controller and neurostimulation device. The system may be implanted in a suitable subject using any convenient approach. Following implantation, the system may be employed to as desired to treat a condition of interest.

It is to be understood that this invention is not limited to particular aspects described, as such may vary. It is also to be understood that the terminology used herein is for the purpose of describing particular aspects only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual aspects described and illustrated herein has discrete components and features which may be readily separated from or combined with the features of any of the other several aspects without departing from the scope or spirit of the present invention. Any recited method can be carried out in the order of events recited or in any other order which is logically possible.

1. An implantable device for target site stimulation, the device comprising:
an integrated control circuit having a controller circuit, a selectable pathway, and a first surface, the controller circuit to alternately enable and disable the selectable pathway, and the selectable pathway to provide electrical power to the surface as directed by the controller circuit; and
an electrode, the electrode coupled to the first surface and to deliver the electrical power received from the selectable pathway to the target site.

2. The implantable device of claim 1, wherein the electrode is convex in shape and extends away from the first surface.

3. The implantable device of claim 1, wherein the electrode covers at least a portion of the first surface.

4. (canceled)

5. The implantable device of claim 1, further comprising a second electrode coupled to the first surface and to deliver the electrical power received from the selectable pathway to the target site.

6. The implantable device of claim 1, further comprising a sensor to acquire biomedical data.

7. The implantable device of claim 1, further comprising
at least one support structure; and
at least one electrode extending from an inside surface of the support structure.

8. The implantable device of claim 1, further comprising a plurality of additional electrodes coupled to the first surface.

9. The implantable device of claim 1, further comprising:
a second surface of the integrated control circuit coupled to selectable pathway; and
a second electrode coupled to the second surface, the second electrode to deliver electrical power to the target site received from the selectable current pathway.

10. The implantable device of claim 9, wherein the second surface is positioned substantively orthogonally to the first surface.

11. The implantable device of claim 9, wherein the second surface is positioned substantively parallel to the first surface.

12. The implantable device of claim 1, wherein the integrated control circuit is hermetically sealed.

13. The implantable device of claim 1, further comprising a metal layer disposed between the first surface and the electrode.

14. The implantable device of claim 13, wherein the metal layer selectively covers the first surface.

15. The implantable device of claim 1, the integrated control circuit further comprising:
a power bus to transfer electrical power from a common conductor to the selectable pathway; and
a device communications bus to receive commands from addressed to the implantable device from a lead communications bus.

16. The device of claim 15, further comprising a metal layer disposed between the first surface and the electrode.

17. The device of claim 16, further comprising an insulative layer disposed between the metal layer and the target site.

18. The implantable device of claim 16, wherein the metal layer selectively covers the first surface.

19. The implantable device of claim 16, wherein the metal layer substantively encloses the integrated control circuit and wherein the device further comprises a first aperture to accept a power bus to deliver electrical power to the integrated control circuit and a second aperture to accept the device communications bus to deliver commands and data to the integrated control circuit.

20. An implantable lead for target site stimulation, the lead comprising:
a common conductor;
a lead communications bus;
a plurality of satellite structures, each satellite structure including:
an integrated control circuit having a controller circuit, a selectable pathway, and a first surface;
the controller circuit coupled with the lead communications bus and with the selectable pathway, and the controller circuit to alternately enable and disable the selectable pathway as directed by commands addressed through and received from the lead communications bus;
the selectable pathway coupled with the common conductor and the selectable pathway to provide electrical power received from the common conductor to the surface as directed by the controller circuit; and
an electrode, the electrode coupled to the first surface and to deliver the electrical power received from the selectable pathway to the target site.

21. A system comprising:
an implantable controller, the implantable controller to provide electrical stimulation pulses;
a common conductor coupled with the implantable controller to receive electrical stimulation pulses; and
a multiplexed, multi-electrode component to provide at least one of neural stimulation and neural sensing, the multiplexed, multi-electrode component comprising two or more individually addressable satellite electrode structures electrically coupled to the common conductor to receive electrical stimulation pulses, wherein each satellite structure comprises an integrated circuit forming at least one electrode, the multiplexed, multi-electrode component operatively coupled to the implantable controller.

22. A method of manufacture comprising the steps of:
providing a substrate;
creating an integrated control circuit upon the substrate to receive electrical stimulation commands and electrical stimulation energy and
creating at least one electrode upon a first surface of the integrated circuit to transfer the electrical stimulation energy to a target site.

23. The method of claim 22, wherein creating the electrode comprises providing a metal layer associated with the first surface.

24. The method of claim 23, further comprising:
creating an insulative layer over the metal layer; and
partially removing the insulative layer to expose a partial top surface area of the metal layer.

25. The method of claim 24, wherein the partial removal of the insulative layer from the metal layer is accomplished by at least one of laser ablation technique or laser electroformimg technique.

26-27. (canceled)

28. The method of claim 23, wherein the step of providing the metal layer comprises cathodic arc deposition.

29. The method of claim 23, wherein the step of creating the electrode comprises exposing a top surface of the electrode.
